(19) United States
(12) Patent Application Publication
(10) Pub. No.: US 2016/0374956 A1
(43) Pub. Date: Dec. 29, 2016

(54) MULTILAYER TYPE PATCH

(71) Applicant: HISAMITSU PHARMACEUTICAL CO., INC., Tosu-shi (JP)

(72) Inventors: Tomohiro SHINODA, Tsukuba-shi (JP); Yasunari MICHINAKA, Tsukuba-shi (JP)

(73) Assignee: HISAMITSU PHARMACEUTICAL CO., INC., Tosu-shi (JP)

(21) Appl. No.: 15/103,449

(22) PCT Filed: Dec. 10, 2014
(86) PCT No.: PCT/JP2014/082726

§ 371 Date: Sep. 15, 2016

(30) Foreign Application Priority Data
Dec. 12, 2013 (JP) 2013-257427
Dec. 24, 2013 (JP) 2013-265780

(51) Int. Cl. A61K 9/70; A61K 47/32; A61K 31/325

(52) U.S. Cl. CPC A61K 9/7084; A61K 31/325; A61K 47/32

(57) ABSTRACT

A multilayer-type patch comprising: a support layer; a drug reservoir layer; and an adhesive layer, wherein
the drug reservoir layer comprises at least one drug selected from the group consisting of rivastigmine and pharmaceutically acceptable salts thereof, and a hydroxy group-containing acrylic-based polymer (A),
the adhesive layer comprises at least one acrylic-based polymer (B) selected from the group consisting of carboxy group-containing alkyl (meth)acrylate copolymer and acrylic acid homopolymer, and a rubber-based adhesive base agent,
a content of the drug in the drug reservoir layer in terms of rivastigmine in free form is 15 to 45% by mass relative to a total mass of the drug reservoir layer, and
a total content of the acrylic-based polymer (B) in the adhesive layer is 7 to 18% by mass relative to a total mass of the adhesive layer.

### TECHNICAL FIELD

The present invention relates to a multilayer-type patch.

### BACKGROUND ART

Transdermally/transmucosally absorbable preparations have been developed as pharmaceutical preparations for administration of drugs into living organisms. Especially, patches have attracted attention, which are easy to handle and which enable an effective blood concentration of a drug to be kept for a long period. For example, rivastigmine (3-[(S)-1-(dimethylamino)ethyl]phenyl methylethylcarbamate), which is a drug for Alzheimer-type dementia, is preferably administered in the form of patch from the viewpoints that loads on the caregivers to manage the administration of the drug can be reduced, and that the drug can be administered continuously. As a patch comprising rivastigmine, for example, International Publication No. WO2013/031992 (PTL 1) describes a patch comprising: a backing; and an adhesive layer containing rivastigmine and an alkyl (meth)acrylate copolymer. However, such a patch (hereinafter, referred to as “single layer-type patch” in some cases) in which the same and single layer serves as a layer containing the drug (a drug reservoir layer) and a layer having adhesiveness (adhesive layer) has a problem in that the adhesiveness to the skin decreases when the drug content is increased for administration for a long period (preferably for 3 to 7 days or longer).

Meanwhile, a pharmaceutical preparation which allows the increase in drug content is a multilayer-type patch comprising a drug reservoir layer containing a drug and an adhesive layer, which are separate layers. As an example of such a multilayer-type patch, Japanese Patent No. 5093545 (PTL 2) describes a patch for treatment of Alzheimer's disease comprising: a backing; a rivastigmine-containing layer containing rivastigmine and an alkyl (meth)acrylate ester resin; an adhesive layer comprising an acrylic-based adhesive agent containing a hydroxy group-containing (meth)acrylic acid ester; and a release liner. Moreover, International Application Japanese-Phase Publication No. 2002-500178 (PTL 3) describes a transdermal device comprising: a backing layer, a rivastigmine-containing layer; a release liner; and a silicone adhesive agent layer, and International Application Japanese-Phase Publication No. 2009-517468 (PTL 4) describes a transdermal therapeutic system comprising: a backing layer; a reservoir layer containing a pharmaceutically active ingredient and a polymer; and an adhesive layer containing a silicone polymer and a tackifier.

### CITATION LIST

### Patent Literature

[PTL 1] International Publication No. WO2013/031992

[PTL 2] Japanese Patent No. 5093545

[PTL 3] International Application Japanese-Phase Publication No. 2002-500178

[PTL 4] International Application Japanese-Phase Publication No. 2009-517468

### SUMMARY OF INVENTION

### Technical Problem

A patch which is desirably attached for a long period, such as a patch containing rivastigmine as a drug, has to gradually and continuously release the drug to the skin to keep the blood concentration of the drug constant for a long period (preferably for 3 to 7 days or longer). Hence, such a patch is especially required to be excellent in sustained-release. The present inventors have found that the single layer-type patch as described in PTL 1 has, however, a problem in that the amount of rivastigmine which can be contained is small as described above, and the sustained-release of rivastigmine from the patch is insufficient, so that rivastigmine is exhausted in a short period. Moreover, PTL 3 states that the use of the silicone adhesive agent in the adhesive layer makes it possible to gradually release the drug. However, the present inventors have found that the multilayer-type patch is still insufficient in terms of the sustained-release of rivastigmine from the patch, and it is difficult for the multilayer-type patch to keep the blood concentration of the drug constant for a long period, because the amount of the drug released and the amount of the drug permeating through the skin increase temporarily and sharply immediately after the patch is attached, and then decrease rapidly.

The present invention has been made in view of the above-described problems of the conventional techniques, and an object of the present invention is to provide a multilayer-type patch which can be attached for a long period and which is excellent in sustained-release of rivastigmine.

### Solution to Problem

The present inventors have conducted intensive study to achieve the above-described object, and consequently have found that a multilayer-type patch comprising: a support layer; a drug reservoir layer; and an adhesive layer, wherein the drug reservoir layer comprises a specific amount of rivastigmine and also a hydroxy group-containing acrylic-based polymer, and the adhesive layer used in combination comprises a specific amount of a carboxy group-containing alkyl (meth)acrylate copolymer and/or acrylic acid homopolymer and a rubber-based adhesive base agent, makes it possible to keep the skin permeation rate of rivastigmine constant for a long period and exhibits an excellent sustained-release. This finding has led to the completion of the present invention.

Specifically, a multilayer-type patch of the present invention is a multilayer-type patch comprising: a support layer; a drug reservoir layer; and an adhesive layer, wherein

the drug reservoir layer comprises at least one drug selected from the group consisting of rivastigmine and pharmaceutically acceptable salts thereof, and a hydroxy group-containing acrylic-based polymer (A),

the adhesive layer comprises at least one acrylic-based polymer (B) selected from the group consisting of carboxy group-containing alkyl (meth)acrylate copolymer and acrylic acid homopolymer, and a rubber-based adhesive base agent,

a content of the drug in the drug reservoir layer in terms of rivastigmine in free form is 15 to 45% by mass relative to a total mass of the drug reservoir layer, and

a total content of the acrylic-based polymer (B) in the adhesive layer is 7 to 18% by mass relative to a total mass of the adhesive layer.

In the multilayer-type patch of the present invention, the acrylic-based polymer (A) is preferably a hydroxyalkyl (meth)acrylate copolymer.

Further, in the multilayer-type patch of the present invention, it is preferable that the adhesive layer comprises the carboxy group-containing alkyl (meth)acrylate copolymer as the acrylic-based polymer (B), it is more preferable that the alkyl (meth)acrylate copolymer contains 0.75 moles or more of carboxy groups per mole of ester groups.

In addition, in the multilayer-type patch of the present invention, the rubber-based adhesive base agent is preferably at least one selected from the group consisting of styrene-isoprene-styrene block copolymer, styrene-butadiene-styrene block copolymer, styrene-butadiene copolymer, polyisobutylene, and polyisoprene.

Moreover, in the multilayer-type patch of the present invention, it is preferable that the adhesive layer has a thickness which results in 100 to 175 g/m². Further, it is preferable that a mass ratio of the drug reservoir layer to the adhesive layer is 10:1 to 1:2.

In the present invention, (meth)acrylic acid refers to acrylic acid and methacrylic acid. Hydroxy group refers to a group represented by “—OH”, and carboxy group refers to a group represented by “—COOH”.

Note that although it is not exactly clear why the present invention can achieve the above-described object, the present inventors speculate as follows. Specifically, in the multilayer-type patch of the present invention, the drug reservoir layer contains the hydroxy group-containing acrylic-based polymer (A) in combination with rivastigmine, and the adhesive layer contains the acrylic-based polymer (B) selected from carboxy group-containing alkyl (meth)acrylate copolymers and acrylic acid homopolymer in combination with the rubber-based adhesive base agent. In the multilayer-type patch of the present invention, this structure creates a difference between the diffusion coefficient and/or the solubility of rivastigmine in the drug reservoir layer and the diffusion coefficient and/or the solubility of rivastigmine in the adhesive layer, so that rivastigmine is rapidly transferred from the drug reservoir layer to the adhesive layer, and also that rivastigmine is gently released from the adhesive layer. The present inventors speculate that, for this reason, the excellent sustained-release is achieved.

### Advantageous Effects of Invention

According to the present invention, it is possible to provide a multilayer-type patch which can be attached for a long period and which is excellent in sustained-release of rivastigmine.

### BRIEF DESCRIPTION OF DRAWINGS

FIG. 1 is a schematic cross-sectional view showing a preferred embodiment of a multilayer-type patch of the present invention.

FIG. 2 is a graph showing the relationship between the skin permeation rate and the measurement time in sustained-release evaluation of patches obtained in Example 1 and Comparative Examples 3 to 5.

FIG. 3 is a graph showing the relationship between the skin permeation rate and the measurement time in sustained-release evaluation of patches obtained in Example 1 and Comparative Examples 3 and 7.

FIG. 4 is a graph showing the relationship between the skin permeation rate and the measurement time in sustained-release evaluation of patches obtained in Example 3 and Comparative Examples 3 and 7.

### DESCRIPTION OF EMBODIMENTS

Hereinafter, a detailed description will be made by showing preferred embodiments of the present invention as examples. The multilayer-type patch of the present invention is a multilayer-type patch comprising: a support layer; a drug reservoir layer; and an adhesive layer, wherein

the drug reservoir layer comprises at least one drug selected from the group consisting of rivastigmine and pharmaceutically acceptable salts thereof, and a hydroxy group-containing acrylic-based polymer (A),

the adhesive layer comprises at least one acrylic-based polymer (B) selected from the group consisting of carboxy group-containing alkyl (meth)acrylate copolymer and acrylic acid homopolymer, and a rubber-based adhesive base agent,

a content of the drug in the drug reservoir layer in terms of rivastigmine in free form is 15 to 45% by mass relative to a total mass of the drug reservoir layer, and

a total content of the acrylic-based polymer (B) in the adhesive layer is 7 to 18% by mass relative to a total mass of the adhesive layer.

(Support Layer)

The support layer according to the present invention is not particularly limited, as long as the support layer can support the drug reservoir layer. Any known support layer for a patch can be employed, as appropriate, as the support layer according to the present invention. Examples of the material of the support layer include synthetic resins such as polyesters including polyethylene terephthalate, polybutylene terephthalate, and polyethylene naphthalate; polyolefins such as polyethylene and polypropylene; polyurethanes; and ethylene-vinyl acetate copolymer, as well as metals such as aluminum, and paper. In addition, the form of the support layer made of such a material is, for example, a film; a sheet such as a foamed sheet, a porous sheet, or a microporous sheet; a fabric such as a woven fabric, a knitted fabric, or a nonwoven fabric; foil; or a laminate of any ones of them. The support layer according to the present invention is preferably impermeable to the drug. Especially, the support layer is preferably a polyester film from the viewpoint that a polyester film has excellent flexibility and excellent drug impermeability. In addition, a thickness of the support layer is not particularly limited, either, and is preferably about 2 to 600 μm, in general.

(Drug Reservoir Layer)

The drug reservoir layer according to the present invention comprises at least one drug selected from the group consisting of rivastigmine and pharmaceutically acceptable salts thereof, and a hydroxy group-containing acrylic-based polymer (A). The drug reservoir layer has a thickness which results in preferably 100 to 500 g/m², and more preferably 150 to 300 g/m². If the thickness of the drug reservoir layer is less than the lower limit, it tends to be difficult to keep a sufficient skin permeation rate and a sufficient sustained-release of rivastigmine for a long period. Meanwhile, if the thickness exceeds the upper limit, the so-called cold flow of the drug reservoir layer becomes more likely to occur, and it tends to be difficult to keep the sufficient thickness and the shape.

[Drug]

The drug reservoir layer according to the present invention comprises rivastigmine as a drug. The rivastigmine may be in a free form (free base) or may be in a form of any of pharmaceutically acceptable salts of rivastigmine. The rivastigmine may be in one of these forms or may be a mixture of two or more of these forms. Examples of the pharmaceutically acceptable salts of rivastigmine include acid addition salts with hydrochloric acid, sulfuric acid, acetic acid, nitric acid, hydrobromic acid, phosphoric acid, methanesulfonic acid, fumaric acid, maleic acid, citric acid, tartaric acid, besylic acid, succinic acid, tannic acid, and the like. Of these forms, rivastigmine in the free form is more preferable in the present invention, from the viewpoint that both the skin permeability and the sustained-release are improved. In the present invention, a sufficient sustained-release is exhibited, even when rivastigmine in the free form, whose skin permeability is high, is used.

In the present invention, the content of rivastigmine in terms of free form in the drug reservoir layer has to be 15 to 45% by mass relative to the total mass of the drug reservoir layer. If the rivastigmine content is less than the lower limit, it is difficult to keep a sufficient skin permeation rate for a long period. Meanwhile, if the rivastigmine content exceeds the upper limit, the plasticity of the drug reservoir layer increases, so that the cohesiveness decreases. Especially when rivastigmine in the free form, which is liquid at normal temperature, is used, this tendency is so strong that the so-called cold flow of the drug reservoir layer becomes more likely to occur, and it is difficult to keep the sufficient thickness and the shape. In addition, the content of rivastigmine in terms of free form in the drug reservoir layer is preferably 20 to 45% by mass and more preferably 20 to 40% by mass relative to the total mass of the drug reservoir layer, from the viewpoints that a more sufficient skin permeation rate tends to be kept for a longer period, and that the thickness and the shape of the drug reservoir layer tend to be more stabilized.

Note that, in the present invention, the content of rivastigmine in terms of free form in the drug reservoir layer refers to a content obtained by converting the total rivastigmine amount including rivastigmine and/or a salt thereof in the drug reservoir layer yet to be stacked on the adhesive layer into the amount of rivastigmine in terms of free form. In addition, in the present invention, the content of each component refers to the content in the drug reservoir layer or the adhesive layer before the adhesive layer and the drug reservoir layer are stacked on each other. In a case where rivastigmine is transferred from the drug reservoir layer to the adhesive layer with the lapse of time, this content refers to that determined assuming that all the rivastigmine contained in the patch is all contained in the drug reservoir layer.

The drug reservoir layer according to the present invention may further comprises drugs other than rivastigmine, within a range not impairing an effect of the present invention. The drug other than rivastigmine is not particularly limited, and examples include hypnotic and sedative drugs (flurazepam hydrochloride, rilmazafone hydrochloride, phenobarbital, amobarbital, and the like), antipyretic and antiinflammatory agents (butorphanol tertrate, perisoxal citrate, acetaminophen, mefenamic acid, diclofenac sodium, aspirin, alclofenac, ketoprofen, flurbiprofen, naproxen, piroxicam, pentazocine, indomethacin, glycol salicylate, aminopyrine, loxoprofen, and the like), steroidal anti-inflammatory agents (hydrocortisone, prednisolone, dexamethasone, betamethasone, and the like), analeptics and stimulants (methamphetamine hydrochloride, methylphenidate hydrochloride, and the like), neuropsychiatric agents (imipramine hydrochloride, diazepam, sertraline hydrochloride, fluvoxamine maleate, paroxetine hydrochloride, citalopram hydrobromide, fluoxetine hydrochloride, alprazolam, haloperidol, clomipramine, amitriptyline, desipramine, amoxapine, maprotiline, mianserin, setiptiline, trazodone, lofepramine, milnacipran, duloxetine, venlafaxine, chlorpromazine hydrochloride, thioridazine, diazepam, meprobamate, etizolam, risperidone, mirtazapin, and the like), hormone drugs (estradiol, estriol, progesterone, norethisterone acetate, metenolone acetate, testosterone, and the like), local anesthetics (lidocaine hydrochloride, procaine hydrochloride, tetracaine hydrochloride, dibucaine hydrochloride, propitocaine hydrochloride, and the like), agents for urinary organs (oxybutynin hydrochloride, tamsulosin hydrochloride, propiverine hydrochloride, tolterodine tartrate, fesoterodine, imidafenacin, and the like), skeletal muscle relaxants (tizanidine hydrochloride, eperisone hydrochloride, pridinol mesylate, suxamethonium chloride, and the like), agents for reproductive organs (ritodrine hydrochloride and meluadrine tartrate), antiepileptic agents (sodium valproate, clonazepam, carbamazepine, and the like), autonomic agents (carpronium chloride, neostigmine bromide, bethanechol chloride, and the like), antiparkinsonian agents (pergolide mesylate, bromocriptine mesylate, trihexyphenidyl hydrochloride, amantadine hydrochloride, ropinirole hydrochloride, talipexole hydrochloride, cabergoline, droxidopa, biperiden, selegiline hydrochloride, and the like), diuretic agents (hydroflumethiazide, furosemide, and the like), respiratory stimulants (lobeline hydrochloride, dimorpholamine, naloxone hydrochloride, and the like), antimigraine agents (dihydroergotamine mesylate, sumatriptan, ergotamine tartrate, flunarizine hydrochloride, cyproheptadine hydrochloride, and the like), antihistamines (clemastine fumarate, diphenhydramine tannate, chlorpheniramine maleate, diphenylpyraline hydrochloride, promethazine, and the like), bronchodilators (tulobuterol hydrochloride, procaterol hydrochloride, salbutamol sulfate, clenbuterol hydrochloride, fenoterol hydrobromide, terbutaline sulfate, isoprenaline sulfate, formoterol fumarate, and the like), cardiotonics (isoprenaline hydrochloride, dopamine hydrochloride, and the like), coronary vasodilators (diltiazem hydrochloride, verapamil hydrochloride, isosorbide mononitrate, nitroglycerin, nicorandil, and the like), peripheral vasodilators (nicametate citrate, tolazoline hydrochloride, and the like), smoking cessation aids (nicotine, and the like), agents for circulatory organs (flunarizine hydrochloride, nicardipine hydrochloride, nitrendipine, nisoldipine, felodipine, amlodipine besylate, nifedipine, nilvadipine, manidipine hydrochloride, benidipine hydrochloride, enalapril maleate, temocapril hydrochloride, alacepril, imidapril hydrochloride, cilazapril, lisinopril, captopril, trandolapril, perindopril erbumine, atenolol, pindolol, bisoprolol fumarate, metoprolol tartrate, betaxolol hydrochloride, timolol maleate, bopindolol malonate, nipradilol, arotinolol hydrochloride, celiprolol hydrochloride, carvedilol, amosulalol hydrochloride, carteolol hydrochloride, bevantolol hydrochloride, terazosin hydrochloride, bunazosin hydrochloride, prazosin hydrochloride, doxazosin mesylate, valsartan, candesartan cilexetil, losartan potassium, clonidine hydrochloride, guanfacine hydrochloride, guanabenz acetate, and the like), antiarrhythmic agents (propranolol hydrochloride, alprenolol hydrochloride, procainamide hydrochloride, mexiletine hydrochloride, nadolol, disopyramide, and the like), anti-malignant-ulcer agents (cyclophosphamide, fluorouracil, tegafur, procarbazine hydrochloride, ranimustine, irinotecan hydrochloride, fluridine, and the like), antilipemic agents (pravastatin, simvastatin, bezafibrate, probucol, and the like), hypoglycemic agents (glibenclamide, chlorpropamide, tolbutamide, glymidine sodium, glybuzole, buformin hydrochloride, and the like), anti-peptic ulcer agents (proglumide, cetraxate hydrochloride, spizofurone, cimetidine, glycopyrronium broimide, and the like), cholagogues (ursodesoxycholic acid, osalmid, and the like), gastroprokinetic agents (domperidone, cisapride, and the like), agents for hepatic diseases (tiopronin and the like), anti-allergic agents (ketotifen fumalate, azelastine hydrochloride and the like), antiviral agents (acyclovir and the like), antivertigo agents (betahistine mesylate, difenidol hydrochloride, and the like), antibiotics (cephaloridine, cefdinir, cefpodoxime proxetil, cefaclor, clarithromycin, erythromycin, methylerythromycin, kanamycin sulfate, cycloserine, tetracycline, benzylpenicillin potassium, propicillin potassium, cloxacillin sodium, ampicillin sodium, bacampicillin hydrochloride, carbenicillin sodium, chloramphenicol, and the like), agents for habitual intoxication (cyanamide and the like), appetite suppressants (mazindol and the like), chemotherapeutic agents (isoniazid, ethionamide, pyrazinamide, and the like), blood coagulation accelerators (ticlopidine hydrochloride, warfarin potassium, and the like), anti Alzheimer's agents (physostigmine, donepezil hydrochloride, tacrine, arecoline, xanomeline, and the like), serotonin receptor antagonist antiemetics (ondansetron hydrochloride, granisetron hydrochloride, ramosetron hydrochloride, azasetron hydrochloride, palonosetron, and the like), antigout agents (colchicine, probenecid, sulfinpyrazone, and the like), narcotic analgesics (fantanyl citrate, morphine sulfate, morphine hydrochloride, codeine phosphate, cocaine hydrochloride, pethidine hydrochloride, and the like). When any of the drugs other than rivastigmine is further blended, a general amount of the drug blended cannot be specified, because it varies depending on the type of the drug and the purpose of the treatment. However, the amount of the drug blended is preferably 20% by mass or less relative to the total mass of the drug reservoir layer from the viewpoints that the cohesiveness of the drug reservoir layer and the skin permeability of rivastigmine are better.

[Acrylic-Based Polymer (A)]

The drug reservoir layer according to the present invention further comprises a hydroxy group-containing acrylic-based polymer (A). In the present invention, an excellent sustained-release of rivastigmine is exhibited by combining the drug reservoir layer comprising the acrylic-based polymer (A) and the rivastigmine with the adhesive layer described in detail later. Note that, in the present invention, each of “acrylic-based polymer (A)” and “hydroxy group-containing acrylic-based polymer (A)” refers to “hydroxy group-containing acrylic-based polymer,” and is contained in the drug reservoir layer.

The acrylic-based polymer (A) has a hydroxy group content of preferably 3 to 30% by mass, and more preferably 5 to 25% by mass. In addition, the acrylic-based polymer (A) is more preferably one containing substantially no carboxy groups from the viewpoint of further improvement in sustained-release of rivastigmine. In the present invention, the expression “containing substantially no carboxy groups” means that the carboxy group content is 3% by mass or less. Note that, when an acrylic-based polymer containing no hydroxy groups but containing carboxy groups as functional groups is used instead of the acrylic-based polymer (A), the sufficient sustained-release of rivastigmine is not exhibited. In addition, the weight average molecular weight of the acrylic-based polymer (A) is not particularly limited, and can be 1,000 to 10,000,000.

Moreover, the acrylic-based polymer (A) is preferably a base agent capable of maintaining the shape of the drug reservoir layer, and is more preferably an adhesive base agent capable of providing adhesiveness to the drug reservoir layer. Note that, in the present invention, the adhesive base agent refers to a compound capable of exhibiting adhesiveness at temperatures (preferably 0° C. to 50° C., more preferably 10° C. to 40° C., and further preferably 15° C. to 40° C.), where the patch is applied.

Examples of the hydroxy group-containing acrylic-based polymer (A) include homopolymers of hydroxyalkyl (meth)acrylates, and hydroxyalkyl (meth)acrylate copolymers, which are copolymers of hydroxyalkyl (meth)acrylates with polymerizable monomers. Examples of the hydroxyalkyl (meth)acrylates include esters obtained from (meth)acrylic acid and hydroxy group-containing alcohols in each of which the alkyl group has 2 to 18 carbon atoms. Specific examples thereof include 2-hydroxyethyl (meth)acrylate, 2-hydroxypropyl (meth)acrylate, 3-hydroxypropyl (meth)acrylate, 4-hydroxybutyl (meth)acrylate, and the like. Specific examples of the polymerizable monomers include (meth)acrylic acid, 2-ethylhexyl (meth)acrylate, methyl (meth)acrylate, butyl (meth)acrylate, vinylpyrrolidone, vinyl acetate, and the like.

Examples of the hydroxy group-containing acrylic-based polymer (A) include 2-hydroxyethyl (meth)acrylate copolymers, 2-hydroxypropyl (meth)acrylate copolymers, 3-hydroxypropyl (meth)acrylate copolymers, 4-hydroxybutyl (meth)acrylate copolymers, and the like. One of these polymers may be used alone or two or more thereof may be used in combination.

In addition, commercially available ones may be used as these acrylic-based polymers (A), and DURO-TAK (registered trademark of Henkel AG &Co. KGaA) 87-202A, DURO-TAK 87-2287, DURO-TAK 87-2516, DURO-TAK 87-2510, DURO-TAK 87-4287, and the like can be used preferably.

In the present invention, the content of the acrylic-based polymer (A) in the drug reservoir layer is preferably 50 to 85% by mass, and more preferably 60 to 80% by mass relative to the total mass of the drug reservoir layer. If the content is less than the lower limit, it tends to be difficult for the drug reservoir layer to contain rivastigmine in such an amount that a sufficient skin permeation rate and a sufficient sustained-release can be kept for a long period. Meanwhile, if the content exceeds the upper limit, the sustained-release of rivastigmine tends to deteriorate.

The drug reservoir layer according to the present invention may further contain additives, such as other adhesive base agent, a tackifier, a softener, a solubilizer, a bulking agent, and a stabilizer in addition to the rivastigmine and/or salt thereof and the acrylic-based polymer (A).

Examples of the other adhesive base agent include rubber-based adhesive base agents, acrylic-based adhesive base agents other than the above-described acrylic-based polymers (A), and silicone-based adhesive base agents. One of these adhesive base agents may be used alone or two or more thereof may be used in combination. When any of the adhesive base agents is contained, the content of the adhesive base agent is preferably 10% by mass or less relative to the total mass of the drug reservoir layer.

Examples of the tackifier include rosin resins, rosin ester resins, terpene resins, terpene phenolic resins, C5-type petroleum resins, C5/C9-type petroleum resins, DCPD (dicyclopentadiene)-type petroleum resins, coumarone-indene resins, alicyclic saturated hydrocarbon resins (hereinafter, abbreviated as “AP” in some cases), and hydrogenated product thereof. One of these tackifiers may be used alone, or two or more thereof may be used in combination. When any of the tackifiers is contained, the content of the tackifiers is preferably 10% by mass or less relative to the total mass of the drug reservoir layer.

The softeners include petroleum-based oils (examples: paraffinic process oils such as liquid paraffin (hereinafter, abbreviated as “LP” in some cases), naphthenic process oils, aromatic process oils, and the like), squalane, squalene, vegetable-based oils (examples: olive oil, camellia oil, castor oil, tall oil, peanut oil, and the like), silicone oils, diprotic acid esters (examples: dibutyl phthalate, dioctyl phthalate, and the like), liquid rubbers (examples: liquid polybutene, liquid isoprene rubber, and the like), liquid fatty acid esters (examples: isopropyl myristate, hexyl laurate, diethyl sebacate, diisopropyl sebacate, and the like), diethylene glycol, polyethylene glycol, glycol salicylate, propylene glycol, dipropylene glycol, triacetin, triethyl citrate, crotamiton, and the like. One of these softeners may be used alone, or two or more thereof may be used in combination. Of these softeners, liquid paraffin, liquid polybutene, isopropyl myristate, diethyl sebacate, and hexyl laurate are preferable, and liquid paraffin is more preferable. When any of the softeners is contained, the content of the softener is preferably 10% by mass or less relative to the total mass of the drug reservoir layer.

Although it depends on the type of the solute to be dissolved, examples of the solubilizers include fatty acids (examples: capric acid, oleic acid, linoleic acid, and the like), fatty acid esters (examples: isopropyl myristate, isopropyl palmitate, and the like), fatty acid derivatives (examples: propylene glycol monolaurate, lauric acid diethanolamide, and the like), glycerin fatty acid esters (examples: glycerin monolaurate, glycerin monooleate, and the like), polyol fatty acid esters (examples: sorbitan monolaurate and the like), aliphatic alcohols (examples: octyldodecanol, isostearyl alcohol, oleyl alcohol, and the like), polyols (examples: propylene glycol, dipropylene glycol, polyethylene glycol, and the like), pyrrolidone derivatives (examples: N-methyl-2-pyrrolidone and the like), organic acids (examples: acetic acid, lactic acid, and the like), and organic acid salts (examples: sodium acetate, sodium lactate, and the like). One of these solubilizers may be used alone, or two or more thereof may be used in combination. When any of the solubilizers is contained, the content of the solubilizer is preferably 10% by mass or less relative to the total mass of the drug reservoir layer.

Examples of the bulking agent include inorganic compounds such as silica, aluminum oxide, aluminum hydroxide, zinc oxide, titanium oxide, talc, clay, kaolin, glass, barium sulfate, calcium carbonate, hydroxyapatite, and ceramics; and organic compounds such as cellulose, silk, polyesters, polyolefins, polyacrylates, polymethacrylates, and polystyrene. One of these bulking agents may be used alone, or two or more thereof may be used in combination.

Meanwhile, examples of the stabilizer include tocopherols, ester derivatives of tocopherols, ascorbic acid, ascorbic acid stearate, nordihydroguaiaretic acid, dibutylhydroxytoluene (hereinafter, abbreviated as “BHT” in some cases), butylhydroxyanisole, and the like. One of these stabilizers may be used alone, or two or more thereof may be used in combination. When any of the bulking agent and/or the stabilizer are contained, the content of each of the bulking agent and the stabilizer is preferably 5% by mass or less relative to the total mass of the drug reservoir layer.

(Adhesive Layer)

The adhesive layer according to the present invention comprises at least one acrylic-based polymer (B) selected from the group consisting of carboxy group-containing alkyl (meth)acrylate copolymers and acrylic acid homopolymer, and a rubber-based adhesive base agent. The adhesive layer has a thickness which results in preferably 50 to 200 g/m², more preferably 100 to 175 g/m², and further preferably 100 to 170 g/m². If the thickness of the adhesive layer is less than the lower limit, it tends to be difficult to keep a sufficient skin permeation rate and a sufficient sustained-release of rivastigmine for a long period. Meanwhile, if the thickness exceeds the upper limit, the so-called cold flow of the adhesive layer becomes more likely to occur, and it tends to be difficult to keep the sufficient thickness and the shape.

[Acrylic-Based Polymer (B)]

The adhesive layer according to the present invention comprises at least one acrylic-based polymer (B) selected from the group consisting of carboxy group-containing alkyl (meth)acrylate copolymer and acrylic acid homopolymer. In the present invention, an excellent sustained-release of rivastigmine is exhibited by the combination of the adhesive layer containing the acrylic-based polymer (B) with the drug reservoir layer. Note that, in the present invention, the “acrylic-based polymer (B)” refers to “carboxy group-containing alkyl (meth)acrylate copolymer and/or acrylic acid homopolymer,” and is contained in the adhesive layer.

The acrylic-based polymer (B) preferably has a carboxy group content of 20 to 63% by mass. If the carboxy group content is lower than the lower limit, the sustained-release of rivastigmine tends to deteriorate. Meanwhile, if the carboxy group content exceeds the upper limit, the skin permeation rate of rivastigmine tends to decrease. In addition, from the viewpoint of further improvement in sustained-release of rivastigmine, the acrylic-based polymer (B) is preferably anionic, and is more preferably one containing substantially no hydroxy groups. In the present invention, the expression “having substantially no hydroxy groups” means that the hydroxy group content is less than 3% by mass. Note that the sufficient sustained-release of rivastigmine is not exhibited, when a cationic acrylic-based polymer having no carboxy groups (for example, a commercially available acrylic-based polymer under the trade name of Eudragit (registered trademark of Evonik Industries AG) RS100, Eudragit RL100, Eudragit E, or Eudragit EPO) is used instead of the acrylic-based polymer (B).

The alkyl (meth)acrylate copolymer is a copolymer of an alkyl (meth)acrylate with a polymerizable monomer, and contains ester groups and carboxy groups. In the present invention, the carboxy group content in the carboxy group-containing alkyl (meth)acrylate copolymer is preferably 0.75 moles or more per mole of ester groups. When the amount of the carboxy groups is less than the lower limit, there is a tendency that the skin permeation rate of rivastigmine cannot be kept constant for a long period, and the sufficient sustained-release is not exhibited. In addition, the carboxy group content is more preferably 0.75 to 2 moles, further preferably 0.9 to 1.1 moles, and particularly preferably 1 mole per mole of ester groups, from the viewpoint that the sufficient skin permeation rate and the sufficient sustained-release of rivastigmine can be kept for a longer period.

In addition, the weight average molecular weight of the alkyl (meth)acrylate copolymer is not particularly limited, and can be 1,000 to 10,000,000.

Examples of the alkyl (meth)acrylate copolymer include 2-ethylhexyl acrylate-vinyl acetate-acrylic acid copolymer, 2-ethylhexyl acrylate-butyl acrylate-acrylic acid copolymer, 2-ethylhexyl acrylate-methyl acrylate-glycidyl methacrylate-acrylic acid copolymer, (meth)acrylic acid-methyl (meth)acrylate copolymer, (meth)acrylic acid-ethyl (meth)acrylate copolymer, and (meth)acrylic acid-butyl (meth)acrylate copolymer. One of these copolymers may be used alone or two or more thereof may be used in combination. In addition, commercially available ones may also be used as these alkyl (meth)acrylate copolymers, and Eudragit (registered trademark of Evonik Industries AG) L100 and the like can be used preferably.

In addition, the weight average molecular weight of the acrylic acid homopolymer is not particularly limited, and can be 1,000 to 10,000,000.

As the acrylic-based polymer (B) according to the present invention, one of these alkyl (meth)acrylate copolymers and acrylic acid homopolymer may be used alone or two or more thereof may be used in combination. From the viewpoint of making the sustained-release easier to control, it is preferable to use one of the alkyl (meth)acrylate copolymers or acrylic acid homopolymer alone. From the viewpoint of making the sustained-release easier to control with a smaller amount, it is preferable to use an alkyl (meth)acrylate copolymer.

In the present invention, the total content of the acrylic-based polymers (B) in the adhesive layer has to be 7 to 18% by mass relative to the total mass of the adhesive layer. If the content is less than the lower limit, or exceeds the upper limit, the sufficient sustained-release of rivastigmine is not exhibited. In addition, the content of the acrylic-based polymer (B) is preferably 8 to 16% by mass relative to the total mass of the adhesive layer, from the viewpoint that a more sufficient skin permeation rate and a more sufficient sustained-release of rivastigmine can be kept for a longer period. In addition, when an alkyl (meth)acrylate copolymer is used as the acrylic-based polymer (B), the content thereof is particularly preferably 8 to 16% by mass relative to the total mass of the adhesive layer. When acrylic acid homopolymer is used, the content thereof is particularly preferably 8 to 16% by mass relative to the total mass of the adhesive layer.

[Adhesive Base Agent]

The adhesive layer according to the present invention further comprises a rubber-based adhesive base agent. The rubber-based adhesive base agent is an adhesive base agent capable of providing adhesiveness to the adhesive layer, and examples thereof include styrene-based block copolymers such as styrene-isoprene-styrene block copolymer (hereinafter, abbreviated as “SIS” in some cases), styrene-butadiene-styrene block copolymer (hereinafter, abbreviated as “SBS” in some cases), styrene-ethylene-butylene-styrene block copolymer, and styrene-butadiene copolymer; natural rubber; polyisobutylene(hereinafter, abbreviated as “PIB” in some cases); and polyisoprene. One of these rubber-based adhesive base agents may be used alone or two or more thereof may be used in combination. Especially, from the viewpoint that a more sufficient skin permeation rate and a more sufficient sustained-release of rivastigmine can be kept for a longer period, the rubber-based adhesive base agent is preferably at least one selected from the group consisting of styrene-isoprene-styrene block copolymer, styrene-butadiene-styrene block copolymer, styrene-butadiene copolymer, polyisobutylene, and polyisoprene, and it is more preferable to use a combination of styrene-isoprene-styrene block copolymer with polyisobutylene.

In the present invention, the content of the rubber-based adhesive base agent in the adhesive layer is preferably 10 to 82% by mass, more preferably 10 to 50% by mass, and further preferably 20 to 40% by mass relative to the total mass of the adhesive layer. If the content is lower than the lower limit, the cohesiveness of the adhesive layer tends to decrease, so that the cold flow becomes more likely to occur. Meanwhile, if the content exceeds the upper limit, it tends to be difficult to exhibit a sufficient adhesiveness. In addition, when a combination of styrene-isoprene-styrene block copolymer with polyisobutylene is used, the mass ratio (SIS:PIB) is preferably 10:1 to 1:5 from the viewpoint that the adhesion of the patch to the skin is further improved.

In addition to the acrylic-based polymer (B) and the rubber-based adhesive base agent, the adhesive layer according to the present invention may further comprise additives such as another adhesive base agent, a tackifier, a softener, a solubilizer, a bulking agent, and a stabilizer, within a range not impairing an effect of the present invention. Especially, it is preferable that at least one selected from the group consisting of tackifiers and softeners be further contained.

Examples of the other adhesive base agent include acrylic-based adhesive base agents other than the acrylic-based polymer (B), and the above-described silicone-based adhesive base agents. One of these adhesive base agents may be used alone or two or more thereof may be used in combination. In the present invention, it is preferable that substantially no acrylic-based adhesive base agent other than the acrylic-based polymer (B) be contained. In the present invention, the expression “substantially no acrylic-based adhesive base be contained” means that the content of the acrylic-based adhesive base agent is 5% by mass or less relative to the total mass of the adhesive layer.

As the silicone-based adhesive agent, it is preferable to use a polymer having an organopolysiloxane backbone. In addition, when the polymer having an organopolysiloxane backbone has hydroxy groups (for example, silanol groups), it is more preferable that at least some of the hydroxy groups be capped with trimethylsilyl groups. In addition, the polymer having an organopolysiloxane backbone further preferably has adhesiveness. Note that the capping with the trimethylsilyl groups includes a mode of end-capping of terminal silanol groups of the polymer having an organopolysiloxane backbone with trimethylsilyl groups. Examples of the polymer having an organopolysiloxane backbone include polydimethylsiloxane (a polymer designated by MQ according to the designation of ASTMD-1418 or the like), polymethylvinylsiloxane (a polymer designated by VMQ according to the designation of ASTMD-1418 or the like), polymethylphenylsiloxane (a polymer designated by PVMQ according to the designation of ASTMD-1418 or the like), and the like. One of these polymers may be used alone, or two or more thereof may be used in combination. In addition, commercially available products such as those of the PSA adhesive agent series (manufactured by Dow Corning Corporation) may also be used as these silicone-based adhesive agents. When any of the silicone-based adhesive agents is contained, the content of the silicone-based adhesive agent is preferably 10% by mass or less relative to the total mass of the adhesive layer. In the present invention, the sufficient skin permeation rate and the sufficient sustained-release of the drug can be kept for a longer period, even when such a silicone-based adhesive base agent is not used.

Examples of the additives such as the tackifier, the softener, the solubilizer, the bulking agent, and the stabilizer are the same as the additives listed for the drug reservoir layer. When the adhesive layer comprises the tackifier, the content thereof is preferably 20 to 50% by mass relative to the total mass of the adhesive layer. In addition, when the adhesive layer comprises the softener, the content thereof is preferably 20 to 50% by mass relative to the total mass of the adhesive layer. Moreover, when the adhesive layer comprises the solubilizer, the content thereof is preferably 10% by mass or less relative to the total mass of the adhesive layer. In addition, when the adhesive layer comprises the bulking agent and/or the stabilizer, the content of each of them is preferably 5% by mass or less relative to the total mass of the adhesive layer.

### (Multilayer-Type Patch)

Hereinafter, a structure of the multilayer-type patch of the present invention will be described in detail with reference to the drawings by showing, as an example, a preferred embodiment of the present invention; however, the present invention is not limited thereto. Note that, in the following description and drawings, the same or equivalent components are denoted by the same reference numerals, and overlapping description thereof is omitted.

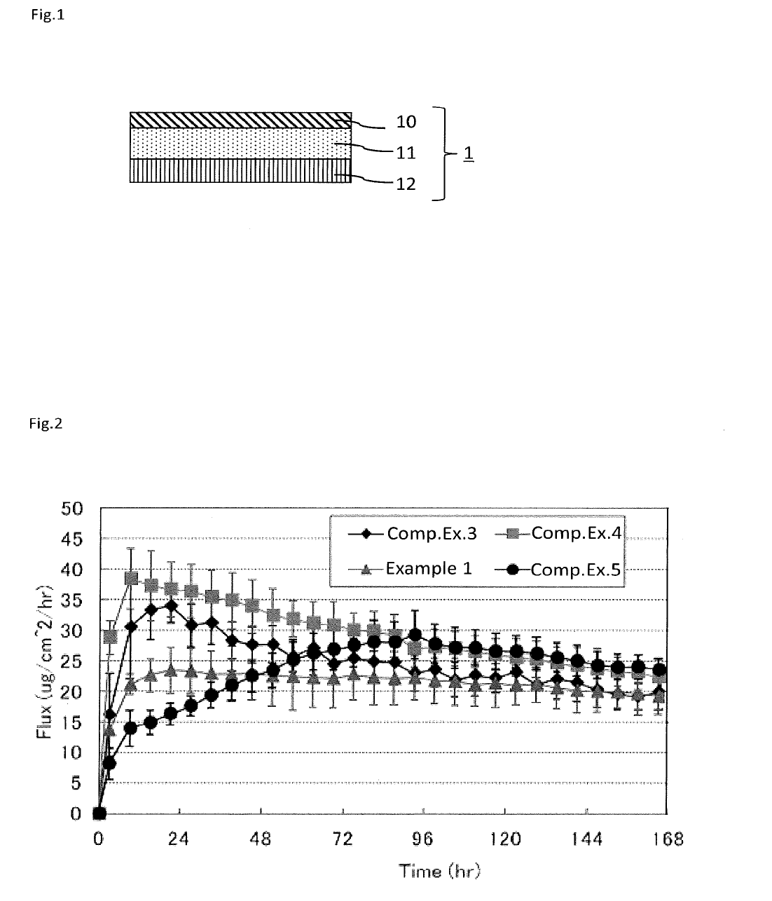

A multilayer-type patch 1 of the present invention preferably has a structure in which a support layer 10, a drug reservoir layer 11, and an adhesive layer 12 are stacked in this order, as shown in FIG. 1. In addition, in the multilayer-type patch 1 of the present invention, for example, an adhesive agent layer or the like may be further stacked between the support layer 10 and the drug reservoir layer 11. In the multilayer-type patch 1 of the present invention, the mass ratio of the drug reservoir layer 11 to the adhesive layer 12 (the mass of the drug reservoir layer 11:the mass of the adhesive layer 12) is preferably 10:1 to 1:2, and more preferably 3:1 to 1:1, from the viewpoint that the sufficient skin permeation rate of rivastigmine tends to be kept for a longer period.

In addition, the multilayer-type patch 1 of the present invention may further comprise a release liner on a surface of the adhesive layer 12 on the side opposite from the drug reservoir layer 11, from the viewpoint of protecting the adhesive layer 12 until use. From the viewpoint of stably fixing the patch 1 to the skin for a longer period, a cover material may be further provided on a surface of the support layer 10 on the side opposite from the drug reservoir layer 11.

When the multilayer-type patch comprises the release liner, the release liner is not particularly limited, and a known release liner for a patch can be employed, as appropriate. The release liner may be a film of a polyester such as polyethylene terephthalate or a film of polyvinyl chloride, polyvinyl idene chloride, or the like; a laminate film of woodfree paper and a polyolefin film; paper; or a laminate of any ones of them. The release liner is preferably subjected to a release treatment such as silicone coating to facilitate the detachment of the release liner. In addition, a thickness of the release liner is not particularly limited, and is preferably about 2 to 300 μm, in general. Further, when the multilayer-type patch comprises the cover material, the cover material is not particularly limited, and a known cover material for a patch can be employed, as appropriate.

Since the drug reservoir layer 11 and the adhesive layer 12 are combined in the multilayer-type the patch 1 of the present invention, the sharp increase and decrease of the amount of rivastigmine released and the amount of rivastigmine permeating through the skin can be sufficiently suppressed, and the skin permeation rate of rivastigmine can be kept constant for a long period, so that an excellent sustained-release can be exhibited. Accordingly, it is possible to keep the blood concentration of rivastigmine constant for a long period (preferably for 3 to 7 days or longer). Note that, in the present invention, the sustained-release of the drug can be evaluated based on, for example, the ratio (Jt/Jmax) between the maximum value (Jmax) of the skin permeation rate of the drug in an attachment period (t) and the skin permeation rate (Jt) after the attachment period has passed. A ratio (Jt/Jmax) closer to 1 is more preferable.

(Method for Producing Multilayer-Type Patch)

The multilayer-type patch of the present invention can be produced by a conventionally known method, without any particular limitation. For example, first, a drug reservoir layer composition containing rivastigmine and/or a salt thereof, the acrylic-based polymer (A), and if necessary, a solvent is prepared, and this composition is applied onto one surface of the support layer to a desired thickness. Then, if necessary, the solvent is removed. Thus, the drug reservoir layer is formed on the one surface of the support layer. Subsequently, an adhesive layer composition containing the acrylic-based polymer (B), the rubber-based adhesive base agent, and, if necessary, a solvent is prepared, and this composition is applied onto the other surface of the drug reservoir layer on the side opposite from the support layer to a desired thickness. Then, if necessary, the solvent is removed. Thus, the adhesive layer is formed. In this manner, the multilayer-type patch of the present invention can be produced. Alternatively, when the multilayer-type patch of the present invention further comprises the release liner, the adhesive layer is formed first by applying the adhesive layer composition onto one surface of the release liner, and the drug reservoir layer is formed by further applying the drug reservoir layer composition. Then, the support layer may be stacked on the surface on the side opposite from the release liner. Alternatively, the adhesive layer formed on one surface of the release liner and the drug reservoir layer formed on one surface of the support layer may be stacked on each other by lamination.

The solvent is not particularly limited, and can be selected, as appropriate, according to the types of the drug and the base agents. Examples of the solvents include lower alcohols such as methanol, ethanol, and isopropanol; toluene, xylene, pentane, n-hexane, cyclohexane, heptane, octane, methyl acetate, ethyl acetate, propyl acetate, methyl butyrate, ethyl butyrate, and propyl butyrate.

### EXAMPLES

Hereinafter, the present invention will be described more specifically on the basis of Examples and Comparative Examples; however, the present invention is not limited to Examples below. Note that the sustained-release evaluation was conducted by the following method in each of Examples and Comparative Examples.

(Sustained-Release Evaluation)

First, a frozen human skin section was thawed at room temperature, and the subcutaneous fat was removed. Then, the skin section was cut to a thickness of approximately 500 μm by using a dermatome to obtain a skin section from which the fat was removed. Subsequently, a patch obtained in each of Examples and Comparative Examples was cut into a 3 cm² piece. After the release liner was removed, this patch was attached to the corneum side of the skin section from which the fat was removed, and the skin section was mounted on a Franz-type flow-through cell, with the dermis side located on the receptor chamber side. While warm water at 32° C. was circulated through an outer peripheral portion of the flow-through cell, a phosphate buffered solution (PBS) was allowed to flow through the receptor chamber at a flow rate of 2.5 ml/hr. A sample liquid was taken out from the receptor chamber every six hours up to 168 hours. For each of the sample liquids taken out, the concentration of the drug (rivastigmine) was quantified by high-performance liquid chromatography, and the skin permeation rate (Flux: μg/cm²/hr) of the drug was calculated from the amount (drug permeation amount) of the drug permeating through the skin section in each period by the following formula:

Flux (μg/cm²/hr)=[drug concentration (μg/ml)×flow rate (ml)]/area of patch (cm²)/time (hr). Moreover, the ratio (J168/Jmax) of the skin permeation rate (J168) 168 hours after the start of the measurement to the maximum value (Jmax) of the skin permeation rate in the measurement time of 168 hours was determined. Note that a patch having a large maximum skin permeation rate (Jmax) of the drug can be considered to be excellent in skin permeability of the drug, and a patch having a J168/Jmax ratio of 0.8 or higher can be considered to be excellent in sustained-release of the drug.

### Example 1

First, a drug reservoir layer composition was obtained by adding 40 parts by mass of rivastigmine (free form) to a solution of an acrylic-based polymer-1 (hydroxy group-containing acrylic-based polymer, acrylic-based polymer (A)) in ethyl acetate (DURO-TAK87-2516 manufactured by Henkel AG & Co. KGaA, acrylic-based polymer-1:60 parts by mass). The obtained drug reservoir layer composition was applied onto one surface of a polyethylene terephthalate film (release liner) having been subjected to a release treatment. Then, ethyl acetate was removed by drying. Thus, a drug reservoir layer having a thickness of 300 g/m² was obtained. Subsequently, the obtained drug reservoir layer was laminated on a polyethylene terephthalate film (support layer) not subjected to a release treatment, and the release liner was removed.

Subsequently, 13.5 parts by mass of styrene-isoprene-styrene block copolymer (SIS, rubber-based adhesive base agent) and 13.5 parts by mass of polyisobutylene (PIB, rubber-based adhesive base agent) were stirred in toluene. To this mixture, 27 parts by mass of liquid paraffin (LP), 36 parts by mass of an alicyclic saturated hydrocarbon resin (AP), and an acrylic-based polymer-2 [alkyl methacrylate copolymer (ester groups:carboxy groups=1:1 (mole ratio)), acrylic-based polymer (B), Eudragit L100, manufactured by Evonik Industries AG] were added. Thus, an adhesive layer composition was obtained. The obtained adhesive layer composition was applied onto one surface of a polyethylene terephthalate film (release liner) having been subjected to a release treatment, and then toluene was removed by drying. Thus, an adhesive layer having a thickness of 150 g/m² was obtained. The obtained adhesive layer was laminated on the surface of the drug reservoir layer on the side opposite from the support layer. Thus, a patch (multilayer-type patch) was obtained in which the release liner, the adhesive layer, the drug reservoir layer, and the support layer were stacked on each other in this order. Table 1 shows the results of sustained-release evaluation conducted on the obtained patch, together with the constitutions of the drug reservoir layer and the adhesive layer.

### Examples 2 and 3

Each patch (multilayer-type patch) was obtained in the same manner as in Example 1, except that the constitutions of the drug reservoir layer and the adhesive layer were changed to the constitutions shown in Table 1. Note that, in Table 1, a polyacrylic acid having a weight average molecular weight of 1,000,000 (acrylic acid homopolymer, acrylic-based polymer (B)) was used as the “polyacrylic acid.” Table 1 shows the results of sustained-release evaluation conducted on each of the obtained patches, together with the constitutions of the drug reservoir layer and the adhesive layer.

### Example 4

A drug reservoir layer was formed in the same manner as in Example 1, except that the content of the acrylic-based polymer-1 in the drug reservoir layer composition was 70 parts by mass, the content of rivastigmine (free form) in the drug reservoir layer composition was 30 parts by mass, and the thickness was 200 g/m². The obtained drug reservoir layer was laminated on a polyethylene terephthalate film (support layer) not subjected to a release treatment, and the release liner was removed. Subsequently, an adhesive layer obtained in the same manner as in Example 1 was laminated on the surface of the drug reservoir layer on the side opposite from the support layer. Thus, a patch (multilayer-type patch) was obtained.

### Comparative Examples 1 and 2

Each patch (single layer-type patch) in which a polyethylene terephthalate film (release liner) having been subjected to a release treatment, a drug reservoir layer, and a support layer were stacked in this order was obtained by forming the drug reservoir layer in the same manner as in Example 1, except that the constitution of the drug reservoir layer was changed to the constitution shown in Table 1, and the thickness was changed to 200 g/m². Note that, in Table 1, a solution (DURO-TAK87-2852 manufactured by Henkel AG & Co. KGaA) of an acrylic-based polymer containing carboxy groups but containing substantially no hydroxy groups in ethyl acetate was used as the “acrylic-based polymer-3” with the ratio of the acrylic-based polymer-3 being the ratio in parts by mass shown in Table 1. Table 1 shows the results of sustained-release evaluation conducted on each of the obtained patches, together with the constitution of the drug reservoir layer.

### Comparative Examples 3 to 7

Each patch (multilayer-type patch) was obtained in the same manner as in Example 1, except that the constitutions of the drug reservoir layer and the adhesive layer were changed to the constitutions shown in Table 1. Table 1 shows the results of sustained-release evaluation conducted on each of the obtained patches, together with the constitutions of the drug reservoir layer and the adhesive layer.

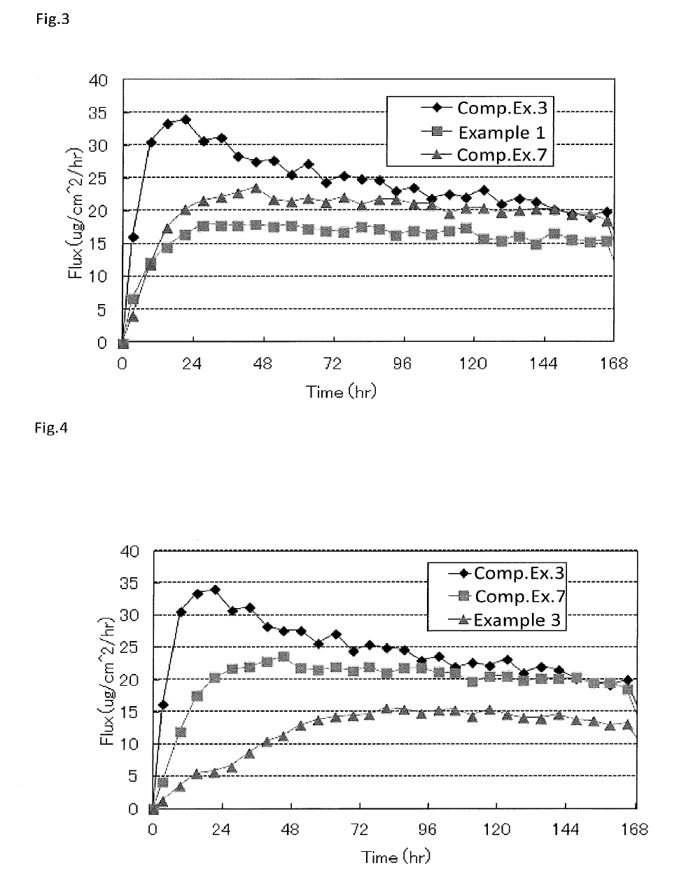

In addition, FIG. 2 shows a graph showing the relationship between the skin permeation rate and the measurement time in the sustained-release evaluation of the patches obtained in Example 1 and Comparative Examples 3 to 5. FIG. 3 shows a graph showing the relationship of the patches obtained in Example 1 and Comparative Examples 3 and 7, and FIG. 4 shows a graph showing the relationship of the patches obtained in Example 3 and Comparative Examples 3 and 7.

TABLE 1

| | | | | | | | | | |
|---|---|---|---|---|---|---|---|---|---|
| | | | | | Comp. | | Comp. | | Comp. |
| Example 1 | Example 2 | Example 3 | Comp. Ex. 1 | Comp. Ex. 2 | Ex. 3 | Comp. Ex. 4 | Ex. 5 | Comp. Ex. 6 | Ex. 7 |

Drug reservoir layer [parts by mass]

| | | | | | | | | | | |
|---|---|---|---|---|---|---|---|---|---|---|
| Rivastigmine | 40 | 40 | 40 | 30 | 30 | 40 | 40 | 40 | 40 | 40 |
| Acrylic-based polymer-1 | 60 | 60 | 60 | — | — | 60 | 60 | 60 | 60 | 60 |
| Acrylic-based polymer-3 | — | — | — | 70 | 65 | — | — | — | — | — |
| Polyvinylpyrrolidone | — | — | — | — | 5 | — | — | — | — | — |

Adhesive layer [parts by mass]

| | | | | | | | | | | |
|---|---|---|---|---|---|---|---|---|---|---|
| SIS | 13.5 | 12.75 | 13.5 | — | — | 15 | 14.25 | 12 | 14.7 | 14.25 |
| PIB | 13.5 | 12.75 | 13.5 | — | — | 15 | 14.25 | 12 | 14.7 | 14.25 |
| LP | 27 | 25.5 | 27 | — | — | 30 | 28.5 | 24 | 29.4 | 28.5 |
| AP | 36 | 34 | 36 | — | — | 40 | 38 | 32 | 39.2 | 38 |
| Acrylic-based polymer-2 | 10 | 15 | — | — | — | — | 5 | 20 | — | — |
| Polyacrylic acid | — | — | 10 | — | — | — | — | — | 2 | 5 |

Sustained-release evaluation

| | | | | | | | | | | |
|---|---|---|---|---|---|---|---|---|---|---|
| Jmax [μg/cm²/hr] | 18.0 | 29.3 | 15.6 | 13.3 | 11.9 | 34.0 | 38.5 | 31.9 | 16.6 | 23.6 |
| J168/Jmax | 0.86 | 0.81 | 0.85 | 0.38 | 0.44 | 0.59 | 0.58 | 0.75 | 0.50 | 0.79 |

Comp. Ex.: Comparative Example

### Comparative Examples 8 to 10

Each patch (multilayer-type patch) was obtained in the same manner as in Example 1, except that the constitutions of the drug reservoir layer and the adhesive layer were changed to the constitutions shown in Table 2. Note that, in Table 2, a methacrylic acid copolymer containing substantially no carboxy groups (Eudragit EPO manufactured by Evonik Industries AG) was used as the “acrylic-based polymer-4,” and a cross-linked polymer of 1-vinyl-2-pyrrolidone was used as the crospovidone. Table shows the results of sustained-release evaluation conducted on each of the obtained patches, together with the constitutions of the drug reservoir layer and the adhesive layer.

TABLE 2

| | | |
|---|---|---|
| Comp. Ex. 8 | Comp. Ex. 9 | Comp. Ex. 10 |

Drug reservoir layer [parts by mass]

| | | | |
|---|---|---|---|
| Rivastigmine | 30 | 30 | 30 |
| Acrylic-based polymer-1 | 70 | 70 | 70 |

Adhesive layer [parts by mass]

| | | | |
|---|---|---|---|
| SIS | 13.5 | 13.5 | 13.5 |
| PIB | 13.5 | 13.5 | 13.5 |
| LP | 27 | 27 | 27 |
| AP | 36 | 36 | 36 |
| Acrylic-based polymer-4 | 10 | — | — |
| Polyvinylpyrrolidone | — | 10 | — |
| Crospovidone | — | — | 10 |

Sustained-release evaluation

| | | | |
|---|---|---|---|
| Jmax [μg/cm²/hr] | 24.0 | 25.5 | 22.5 |
| J168/Jmax | 0.40 | 0.41 | 0.43 |

Comp. Ex.: Comparative Example

As is apparent from the results shown in Tables 1 and 2 and FIGS. 2 to 4, the sufficient skin permeation rate of rivastigmine was kept constant for a long period (7 days) in each of the multilayer-type patches of the present invention, demonstrating that the multilayer-type patches of the present invention were excellent in sustained-release of rivastigmine. In addition, it was found that the patch obtained in Example 4 was also excellent in sustained-release of rivastigmine as in the case of the patch obtained in Example 1.

### INDUSTRIAL APPLICABILITY

As described above, according to the present invention, it is possible to provide a multilayer-type patch which can be attached for a long period and which is excellent in sustained-release of rivastigmine.

### REFERENCE SIGNS LIST

1: multilayer-type patch
10: support layer
11: drug reservoir layer
12: adhesive layer.

1. A multilayer-type patch, comprising:
a support layer;
a drug reservoir layer; and
an adhesive layer,
wherein the drug reservoir layer comprises at least one drug selected from the group consisting of rivastigmine and pharmaceutically acceptable salts thereof, and a hydroxy group-containing acrylic-based polymer, the adhesive layer comprises at least one acrylic-based polymer selected from the group consisting of carboxy group-containing alkyl (meth)acrylate copolymer and acrylic acid homopolymer, and a rubber-based adhesive base agent, a content of the drug in the drug reservoir layer in terms of rivastigmine in free form is 15 to 45% by mass relative to a total mass of the drug reservoir layer, and a total content of the acrylic-based polymer in the adhesive layer is 7 to 18% by mass relative to a total mass of the adhesive layer.

2. The multilayer-type patch according to claim 1, wherein the hydroxy group-containing acrylic-based polymer is a hydroxyalkyl (meth)acrylate copolymer.

3. The multilayer-type patch according to claim 1, wherein the adhesive layer comprises the carboxy group-containing alkyl (meth)acrylate copolymer.

4. The multilayer-type patch according to claim 3, wherein the alkyl (meth)acrylate copolymer contains 0.75 moles or more of carboxy groups per mole of ester groups.

5. The multilayer-type patch according to claim 1, wherein the rubber-based adhesive base agent is at least one selected from the group consisting of styrene-isoprene-styrene block copolymer, styrene-butadiene-styrene block copolymer, styrene-butadiene copolymer, polyisobutylene, and polyisoprene.

6. The multilayer-type patch according to claim 1, wherein the adhesive layer has a thickness which results in 100 to 175 g/m².

7. The multilayer-type patch according to claim 1, wherein a mass ratio of the drug reservoir layer to the adhesive layer is 10:1 to 1:2.

8. The multilayer-type patch according to claim 2, wherein the adhesive layer comprises the carboxy group-containing alkyl (meth)acrylate copolymer.

9. The multilayer-type patch according to claim 8, wherein the alkyl (meth)acrylate copolymer contains 0.75 moles or more of carboxy groups per mole of ester groups.

10. The multilayer-type patch according to claim 2, wherein the rubber-based adhesive base agent is at least one selected from the group consisting of styrene-isoprene-styrene block copolymer, styrene-butadiene-styrene block copolymer, styrene-butadiene copolymer, polyisobutylene, and polyisoprene.

11. The multilayer-type patch according to claim 2, wherein the adhesive layer has a thickness which results in 100 to 175 g/m².

12. The multilayer-type patch according to claim 2, wherein a mass ratio of the drug reservoir layer to the adhesive layer is 10:1 to 1:2.

13. The multilayer-type patch according to claim 3, wherein the rubber-based adhesive base agent is at least one selected from the group consisting of styrene-isoprene-styrene block copolymer, styrene-butadiene-styrene block copolymer, styrene-butadiene copolymer, polyisobutylene, and polyisoprene.

14. The multilayer-type patch according to claim 3, wherein the adhesive layer has a thickness which results in 100 to 175 g/m².

15. The multilayer-type patch according to claim 3, wherein a mass ratio of the drug reservoir layer to the adhesive layer is 10:1 to 1:2.

16. The multilayer-type patch according to claim 4, wherein the rubber-based adhesive base agent is at least one selected from the group consisting of styrene-isoprene-styrene block copolymer, styrene-butadiene-styrene block copolymer, styrene-butadiene copolymer, polyisobutylene, and polyisoprene.

17. The multilayer-type patch according to claim 4, wherein the adhesive layer has a thickness which results in 100 to 175 g/m².

18. The multilayer-type patch according to claim 4, wherein a mass ratio of the drug reservoir layer to the adhesive layer is 10:1 to 1:2.

19. The multilayer-type patch according to claim 5, wherein the adhesive layer has a thickness which results in 100 to 175 g/m².

20. The multilayer-type patch according to claim 5, wherein a mass ratio of the drug reservoir layer to the adhesive layer is 10:1 to 1:2.
